(19) United States
(12) Patent Application Publication
(10) Pub. No.: US 2026/0002845 A1
(43) Pub. Date: Jan. 1, 2026

(54) Submersible Testing Units For Testing / Sampling Bodies of Water, and Related Systems, Methods, and Software

(71) Applicant: University of Vermont and State Agricultural College, Burlington, VT (US)

(72) Inventors: Tian Xia, Burlington, VT (US); Soheyl Faghir Hagh, Burlington, VT (US); Parmida Amngostar, Burlington, VT (US); Dryver Huston, South Burlington, VT (US); Dylan Burns, Burlington, VT (US)

(21) Appl. No.: 19/250,934

(22) Filed: Jun. 26, 2025

Related U.S. Application Data
(60) Provisional application No. 63/665,055, filed on Jun. 27, 2024.

(51) Int. Cl. G01N 1/10; B64D 1/12; G01N 33/18

(52) U.S. Cl. CPC G01N 1/10; B64D 1/12; G01N 33/1886

(57) ABSTRACT

Submersible testing units (STUs) that each include either one or more sample chambers for acquiring one or more samples, e.g., from a body of water, or one or more submersible sensors for acquiring sensor data, e.g., from the body of water, or both one or more sample chambers and one or more submersible sensors. In some embodiments, an STU includes multiple sample chambers that can be opened and closed independently of one another. In some embodiments, an STU having one or more submersible sensors is part of an STU deployment system that transmits sensor data to a data-collection node in real time while the one or more submersible sensors are submerged. Related components, systems, methods, and software are also disclosed.

### RELATED APPLICATION DATA

The present application claims the benefit of priority of U.S. Provisional Patent Application Ser. No. 63/665,055 filed on Jun. 27, 2024, and titled “SUBMERSIBLE TESTING UNITS FOR TESTING/SAMPLING BODIES OF WATER, AND RELATED SYSTEMS, METHODS, AND SOFTWARE”, which is incorporated into the present application by reference in its entirety.

### GOVERNMENT RIGHTS

This invention was made with government support under Award G22AP00025-00 issued by the United States Geological Survey and Grant 2119485 awarded by the U.S. National Science Foundation. The government has certain rights in the invention.

### FIELD

The present disclosure generally relates to the field of water testing. In particular, the present disclosure is directed to submersible testing units for testing/sampling bodies of water, and related systems, methods, and software.

### BACKGROUND

Testing and monitoring of geographical bodies of water are important endeavors for ensuring that human activities, such as mining, farming, manufacturing, using various forms of transportation, energy producing, etc., and polluting the environment in general, do not irreparably degrade those bodies of water. For example, harmful algal blooms (HABs) pose a significant threat to coastal and inland water bodies due to the production of a suite of toxins that are hazardous to aquatic ecosystems and public health. Human poisoning can occur through the consumption of contaminated seafood due to toxin accumulation in shellfish in marine systems or direct ingestion of fish and contaminated water sources in freshwater. Beyond direct public health impacts, HABs and Cyanobacteria HABs (cHABs) have economic ramifications for coastal and lakeside communities. In addition to dangerous toxin production, these blooms consume oxygen through high biomass production and decomposition, which affects fish habitat, and creates unpleasant taste and odor compounds. In freshwater, cyanobacteria blooms appear to be increasing globally, driven by the interactive effects of climate change and eutrophication. In some cases, this is due to agricultural fertilizers, chemical discharge of industrial waste, or urban sewage and waste runoff. However, there is evidence that cHABs are increasingly common in remote, low-nutrient and cold-water ecosystems requiring novel sampling and measurement approaches.

Predictions indicate that by the year 2090, the average number of days with cHABS will rise from approximately 7 days per waterbody to 18-39 days, exacerbating their impacts. To assess and mitigate cyanobacteria blooms promptly, novel early detection methods and prevention strategies such as water quality regulations are imperative.

### SUMMARY

In one implementation, the present disclosure is directed to submersible testing unit (STU) system for conducting testing of a body of water. The STU system includes an STU deployment system that includes: an STU having a submersible portion designed and configured for being submerged in the body of water; a sensor system having at least one sensor deployed on the submersible portion of the STU for sensing a first parameter of the water in the body of water when the at least one sensor is submerged in the body of water, wherein the sensor system generates sensor data that includes first data regarding the first parameter; an unmanned aerial vehicle (UAV) coupled to the STU, wherein the UAV is designed and configured to be controlled to move the STU to one or more geographic locations on the body of water and to submerge the submersible portion of the STU in the body of water; a positioning system configured for determining a location of the UAV during use of the STU deployment system; and a wireless communications system operatively coupled to the sensor system, wherein the wireless communications system is configured to transmit the sensor data offboard of the STU deployment system.

In another implementation, the present disclosure is directed to a submersible testing unit (STU) for testing a body of water. The STU includes a main body designed and configured to be at least partially submerged in the body of water; a sampling system supported by the main body and that includes a plurality of sampling chambers designed and configured to receive a sample of the water in the body of water when the inlet is submerged in the water; a sealing system, in operable communication with the opening, having a sealing member designed and configured to seal the inlet after receiving the sample of water, the sealing member having a sealed position and an unseal position; and a seal-actuation system operatively coupled to the sealing member and designed and configured to move the sealing member between the sealed and unsealed positions.

In yet another implementation, the present disclosure is directed to a method of testing a body of water. The method includes receiving sensor data from one or more submersible sensors when each of the one or more submersible sensors is submerged in the body of water at a first testing location; and wirelessly transmitting the sensor data to a data-collection node while the one or more submersible sensors remains submerged in the body of water at the first testing location.

In still yet another implementation, the present disclosure is directed to a machine-readable storage medium containing machine-executable instructions for performing the method that includes the above method.

### BRIEF DESCRIPTION OF THE DRAWINGS

For the purpose of illustration, the accompanying drawings show aspects of one or more embodiments of the disclosure. However, it should be understood that the scope of this disclosure is/are not limited to the precise arrangements and instrumentalities shown in the drawings, wherein:

FIG. 1 is a high-level block diagram illustrating an example submersible testing unit (STU) system made in accordance with the present disclosure;

FIG. 2A is a perspective view of an example STU made in accordance with aspects of the present disclosure and including a seal-actuation mechanism associated with each of the sample chambers, showing each of sealing members in its sealing position;

FIG. 2B is a perspective view of the STU of FIG. 2A, showing all of the sealing members in non-sealing positions;

FIG. 3A is an isometric view of another example STU made in accordance with aspects of the present disclosure and including a single sealing member and a single seal-actuation mechanism, showing the sealing member in a position in which one of the sealing chambers is unsealed;

FIG. 3B is an exploded isometric view of the example STU of FIG. 3A, showing the motor of the seal-actuation mechanism;

FIG. 3C is an enlarged view of the sealing side of the sealing member of FIGS. 3A and 3B:

FIG. 4A is an isometric view of a further example STU made in accordance with aspects of the present disclosure and including a plurality of sealing members and a single seal-actuation mechanism, showing one of the sealing members being actuated to an open position while the remaining sealing members remain in their sealing positions;

FIG. 4B is an enlarged exploded isometric view of the example STU of FIG. 4A;

FIG. 4C is a further enlarged side view of one of the caps of the sample-chamber structures of the STU of FIGS. 4A and 4B;

FIG. 4D is a bottom isometric view of the cap of FIG. 4C;

FIG. 5A is a high-level schematic diagram of a sensor board for an example implementation of an STU of the present disclosure;

FIG. 5B is a flow diagram illustrating a process of geographic information (GIS) mapping used in the example STU implementation;

FIG. 5C is a high-level block diagram of web integration used with the example STU implementation;

FIG. 6 is a timing diagram illustrating signal timings of various components of the STU in the example STU implementation; and

FIG. 7 is a flow diagram illustrating the operation of the STU deployment system of the example STU implementation.

### DETAILED DESCRIPTION

The entire contents of the appended claims are incorporated into this Detailed Description section by reference and should be treated as if originally presented herein.

Unless noted otherwise, the modifiers “first”, “second”, “third”, “fourth”, and the like, do not denote any particular order or importance, location, priority, etc. Rather, these modifiers are used simply to differentiate elements that are the same as or similar to one another in a set of two or more of such elements.

### GENERAL

In some aspects, the present disclosure is directed to a submersible testing unit (STU) system for conducting testing and/or sampling of a body of water, such as a lake, a pond, a swamp, a bay, a reservoir, a canal, a fjord, and an estuary, among others. Examples of tests that an STU system of the present disclosure may be configured to perform or participate in include, but are not limited to: in-situ testing that utilized one or more submersible sensors, such as, but not limited to, a temperature sensor, a total-dissolved-solids (TDS) sensor, a pH sensor, and a turbidity sensor, a depth sensor, a dissolved oxygen (O₂) sensor, carbon dioxide (CO₂) sensor, a nutrient sensor, a fluorometer, and heavy metal sensors, among others, and any combination thereof; testing that requires acquiring at least one sample of water from the body of water; and testing requires acquiring one or more types of images, such as visible light images, near-infrared (NIR) light images, etc., and any combination of such types of testing. For example, in some embodiments, an STU system of the present disclosure may support in-situ submersible-sensor testing, sample-based testing, and image-based testing. In some embodiments, an STU system of the present disclosure may support only submersible-sensor testing, or only sample-based testing, or a combination of submersible-sensor testing and sample-based testing, among other variations. In some embodiments, in-situ testing may be performed in real time, for example, to provide instant feedback data for use by a human operator and/or a component of the STU system to make decisions about adjusting a current testing/sampling mission in real time due to trends, anomalies, or other noteworthy characteristics of the real-time data.

When the STU system supports sample-based testing, the STU includes one or more sample chambers for collecting water sample(s) for offboard analysis. Examples of offboard analysis include, but are not limited to, analysis techniques that utilize one or more reagents for testing chemical composition, utilizing one or more fluorophores for testing sample content, and/or other techniques, such as centrifuging, among others. In many embodiments, the STU includes a plurality of sample chambers, such as 2 sample chambers to 24 sample chambers, or more. In some embodiments, a benefit of using multiple discrete sample chambers is the avoidance cross contamination problems in other water sensing systems that use a common tube for water collection. The STU system may utilize the plurality of sample chambers in any one or more manners, in some cases, depending on the mission at issue, with the STU being flexible in the manner by which it utilizes the plurality of chambers. For example, one manner in which the STU system may utilize the multiple chambers is to use the chambers to collect one sample at each of a plurality of locations relative to the body of water. For example, if the STU includes 4 sample chambers, the first sample chamber may be used to obtain a first water sample at a first location, the second sample chamber may be used to obtain a second water sample at a second location different from the first location, the third sample chamber may be used to obtain a third water sample at a third location different from each of the first and second locations, and the fourth sample chamber may be used to obtain a fourth water sample at a fourth location different from each of the first, second, and third locations.

In some cases the offboard testing requirements may dictate that more than one sample per location be acquired. Using the same four-location example but wherein each location needs two samples, the STU system may include 8 sample chambers, with both of sample chamber 1 and 2 obtaining samples at the first location, both of sample chambers 3 and 4 obtaining samples at the second location, both of sample chambers 5 and 6 obtaining samples at the third location, and both of sample chambers 7 and 8 obtaining samples at the fourth location. In another example, some locations may require multiple samples, whereas other locations may need only one sample. For example, for the 8-sample-chamber, four-location example just given but wherein each of the first and second locations require 3 samples and each of the third and fourth location require only one sample, each of sample chambers 1-3 may obtain samples at the first location, each of sample chambers 4-6 may obtain samples at the second location, sample chamber 7 may obtain a sample at the third location, and sample chamber 8 may obtain a sample at the fourth location. Software running onboard the STU system may automatically keep track of the testing locations and the relationships between the sample chambers and the testing locations. Examples of how one or more sample chambers can be integrated into an STU system of the present disclosure are described in detail below.

In some embodiments of an STU system of the present disclosure that includes one or more submissible sensors, the STU system may include an STU deployment system that comprises an uncrewed aerial vehicle (UAV), such as a hoverable drone, and an STU that includes a submersible portion that includes the one or more submersible sensors. In some examples, such an STU system may be configured to provide real-time or near-real-time sensor data to one or more locations offboard of the STU deployment system. For example, the STU system may include a wireless data link between the STU deployment system and a data-collection node, which may be, for example, a wireless access point connected to a network, such as a network that includes a wide-area network (WAN), the Internet, and/or a cellular network, among others, or may be a control console for the STU system, among other things. In some embodiments, the wireless data link may comprise a long range WAN (LoRaWAN) based on non-licensed frequencies or may be based on one or more other wireless data link technologies, such as, but not limited to, ZIGBEE®, WI-FI®, BLUETOOTH®, LTE, 5G/6G, and STARLINK® technologies, among others.

In some embodiments of an STU system of the present disclosure that use a network-connected wireless-access point, or other connection to a network, may provide one or more features via the connected network. For example, sensor data obtained from the STU via one or more submersible sensors can be stored using the network, such as on one or more STU-system servers or a cloud-based storage solution. As another example, STU-system software may be provided via the network, such as via one or more dedicated servers or via a cloud-based service, among other architectures. STU-system software may provide any one or more functions, such as, for example, 1) a dashboard to the sensor data, offboard testing data, multi-spectral imaging data, mission information (e.g., time of data-capture, location(s) of data capture, map information, historical data, etc.), and/or any other information that may relevant to the body of water at issue, 2) a mission interface that allows a user to create and/or schedule a testing mission for deploying one or more STU deployment systems to one or more bodies of water, and/or 3) a control interface that provides a soft control console that allows a user to remotely control any or all aspects of an STU system of the present disclosure and, if applicable, an uncrewed delivery vehicle, such as a relatively large airborne vehicle, an overland vehicle, or a waterborne vehicle, that can be remotely controlled to deliver one or more STU deployment systems to a location proximate to or onto the body of water under investigation so that the STU deployment system(s) can be put into service for that body of water.

As noted above, in addition to any submersible sensor(s) that may be present on the STU, if any, an STU of the present disclosure may include one or more sample chambers that allow the STU to acquire, correspondingly, one or more samples of the water from the body of water being tested. Each sample chamber has an inlet that, when submerged in a body of water and open to the water, allows water to flow into the sample chamber as a water sample. In some embodiments, the sample chamber may be provided by, for example, a conventional sample tube (e.g., a centrifuge tube, test tube, etc.) of any suitable size (e.g., 15 ml, 25 ml, 50 ml, etc.) or by another structure of any suitable size and shape. In some embodiments, the structure that defines the sample chamber, i.e., the “sample-chamber structure”, is removably secured to the corresponding STU. This allows for easily swapping out sample chambers during a mission, for example, to replace a full sample chamber with a fresh one, to swap out a damaged sample-chamber structure, or to change the size of a sample chamber, among other things.

Typically, the inlet to a sample chamber will be a single opening. However, in other embodiments the inlet may be defined by two or more openings. Typically, the inlet will be sealed at all times except when collecting a water sample. As described and exemplified below, the inlet may be sealed via a sealing member of any of a variety of types. In some embodiments, the sealing member is unique to a particular inlet and corresponding sample chamber. In some embodiments that have multiple sample chambers and multiple corresponding inlets, the sealing member is common to at least one other of the multiple inlets and corresponding sample chambers.

In some embodiments, the sealing member is movable by a suitable seal-actuation mechanism, which can include, for example, a linear actuator (e.g., linear motor, pneumatic piston, hydraulic piston, etc.) or a rotational motor (e.g., a stepper motor, a servomotor, etc.), either alone or in combination with one or more other components, such as a gearbox and/or motion converter (e.g., a rack and pinion assembly, helical gear, etc.). Examples of seal-actuation mechanisms that can be used to move a sealing member are described and exemplified below.

Since many STUs of the present disclosure will have more than one sample chamber, and since an STU having only a single sample chamber can be made using components and techniques the same as or similar to any one or more of the example multi-sample-chamber embodiments disclosed herein, only multi-sample-chamber embodiments are addressed in detailed in this disclosure.

In some embodiments, the STU may comprise a main body that holds each of the submersible sensors that may be present on the STU and/or that holds each of the sample-chamber structures that may be present on the STU, along with any necessary support system(s), such as one or more sealing members, one or more seal-actuation mechanism, and any local support electronics needed for the submersible sensor(s) and/or the seal-actuation mechanism(s), among other things. In some embodiments, the local support electronics may include one or more microprocessors and memory for controlling the functioning of the submersible sensor(s), the seal-actuation mechanism(s), and/or any other controllable component onboard the STU. Any control methods needed to operate an STU of the present disclosure, such as methods of moving one or more sealing members and method of controlling operation of any submersible sensor, etc., may be performed by any suitable combination of hardware and software. Examples of hardware include, but are not limited to, one or more processors of any suitable type (e.g., FPGA, general purpose, ASIC, system on chip, custom chip, etc.) and memory of any one or more types (e.g., RAM, ROM, cache, persistent, magnetic, bubble, etc.), with the memory storing machine-executable instructions encoding the method(s). As used herein and in the appended claims, the term “machine-readable storage medium” denotes hardware memory of any one or more types and does not include transitory signals, such as digital information encoded onto a carrier wave or into a pulsed signal.

In some embodiments, the STU deployment system may include a semi-autonomous navigation controller that receives gross navigation commands, such as “proceed to location X”, with the semi-autonomous navigation controller using one or more onboard navigation sensors, such as a positioning sensor (e.g., a GPS sensor) and an inertial measurement unit, among others, to auto-navigate to location X. In some embodiments, the STU deployment system may include an autonomous navigation controller that can seek and find the first navigation locations. For example, an autonomous navigation controller may be programmed to seek and find a first testing/sampling location that may be inputted into an autonomous navigation controller using any suitable local or global coordinate system, such as a GPS coordinate system, among others. Those skilled in the art will readily appreciate the variety of ways to cause a mobile sensing device to navigate to a desired first measurement location, with the foregoing examples being just a few of such ways.

In some embodiments, autonomous navigation may be based on sensor data that the acquired by one or more submersible sensors and/or images acquired by one or more imaging devices aboard the STU deployment system. For example, after acquiring sensor data at multiple locations, the data may indicate a directionality trend for a parameter, and that directionality may be used to determine a location of a next testing/sampling location.

In some embodiments, communication of sensor data, control signals, control commands, etc., may be performed either wirelessly or wiredly, or a combination of wirelessly and wiredly. In some embodiments, the STU is tethered to a UAV via a tether. In such embodiments, the tether may include one or more communications cables. In a tethered embodiment, power to operate the STU may be provided from the UAV to the STU via the tether. In other embodiments, the STU may be powered by one or more power sources (e.g., one or more batteries) located onboard the STU. In some embodiments, wireless communications between the STU and UAV or between the STU and a remote device, such as a wireless access point to a network or a remote workstation, may be provided using, for example, any suitable radio technology, such as the LoRaWAN or other technology noted above. In some embodiments, wireless communications between the STU deployment system and a remote device may be handled aboard the UAV instead of the STU itself.

In some embodiments, the main body of the STU is attached directly to a tether that, in turn, is attached to a UAV, either with a fixed-length tether or a variable-length tether, with or without a safety breakaway connector to avoid losing the UAV if, for example, the STU gets entangled is something in the body of water. However, in other embodiments, the main body may be removably secured to a base that is attached to the tether. Depending on the nature of the STU, the base may provide only a mechanical attachment for the main body of the STU, or it may provide both a mechanical attachment and one or more electrical connections that pass electrical power and/or signals between the tether and the main body of the STU, and/or it may include some or all of the submersible sensors, and/or it may include all of the electronics for the STU. Having the main body removably secured to the base allows a user to swap out the main body of the STU for any one or more purposes, such as to provide a fresh set of sample chambers, to change the sensing and/or sampling capabilities of the STU (e.g., different main bodies may be provided with different submersible sensor sets and/or differing numbers of sample chambers, etc.), and/or to replace a damaged main body. As noted above, in some embodiments the base may hold the submersible sensors and all electronics and the actuator(s) for the STU, such that the main body supports only a plurality of sample-chamber structures, and, in some embodiments, the sealing member(s). Those skilled in the art will readily appreciate the wide variety of variants that can be made using only the present disclosure as a guide.

The foregoing and other embodiments are exemplified in the following section.

### EXAMPLE EMBODIMENTS

With the foregoing in mind, this section describes some example embodiments that combine various features, elements, and components discussed above. These examples are not intended to cover all possible combinations and permutations of the features, elements, and components discussed above. Rather, they are simply illustrative of manners in which the foregoing features, elements, and component can be combined with one another and results that can be achieved therefrom.

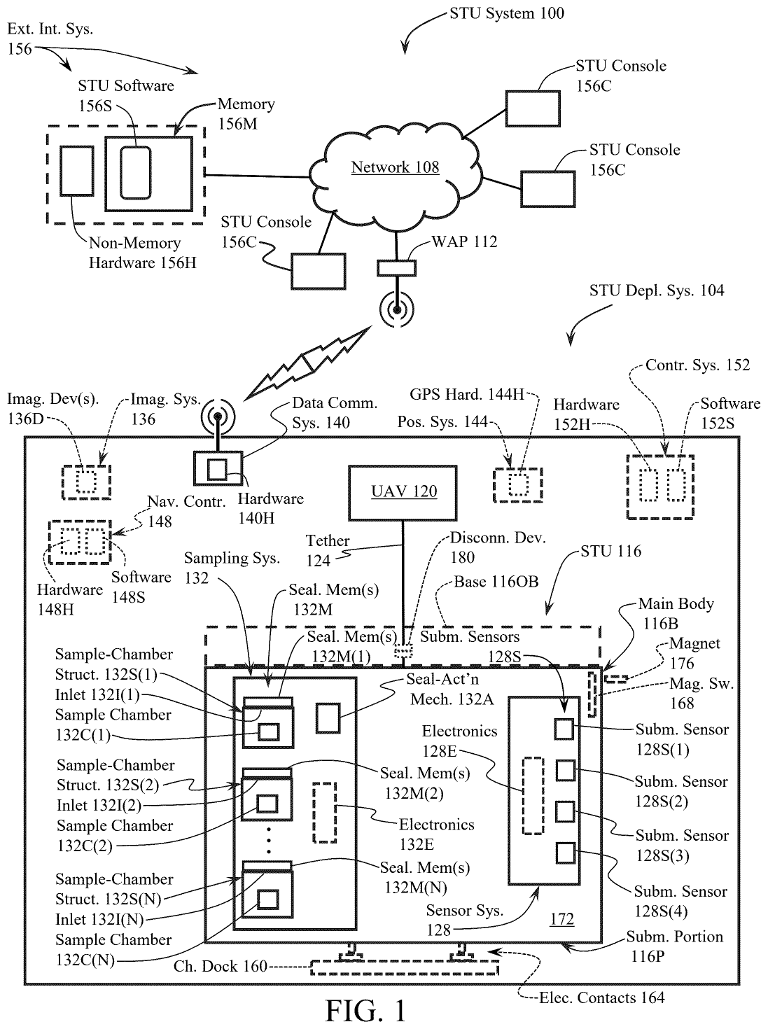

FIG. 1 shows an example STU system 100 made in accordance with various aspects of the present disclosure. In this example, the STU system 100 includes an STU deployment system 104 that is wirelessly connected to a network 108, here, via a Wireless Access Point (WAP) 112. This embodiment is particularly configured for various functionalities to be provided by or otherwise supported via the network 108. The network 108 in this example represents any and all network types needed to effect the requisite functionalities of the STU system 100. Examples of such network types that the network 108 represents include, but are not limited to, the Internet, one or more cellular communications networks, and one or more WANs, among others, that provide end-to-end connectivity between the WAP 112 and any relevant device across the network, such as, but not limited to, a server, server farm, or other network-connected device. In some embodiments, the WAP 112 may be considered a data-collection node as it is a node that receives data from the STU deployment system 104, as discussed below. It is noted, however, that in other embodiments, a data-collection node may be another device, such as, for example, a non-network connected device, such as a STU console (not shown) that is wirelessly connected to the STU deployment system 104.

In this example, the STU deployment system includes an STU 116 and a UAV 120, with the STU being physically connected to the UAV, such as via a tether 124. While a tether is shown, it is noted that in other embodiments the physical connection may be direct or via a rigid member. Depending on the locations of the various electronic and electrical components as between the UAV 120 and the STU 116 and whether communications with the STU are handled wiredly or wirelessly, the tether 124 may or may not include one or more electrical wires and/or one or more communications cables. The UAV 120 may be any suitable UAV, such as a multirotor hoverable drone of any suitable size. As a nonlimited illustrative embodiment, the UAV 120 may be an AURELIA® X4 drone available from Aurelia Technologies, Inc., Las Vegas, Nevada, which has a payload capacity of 1.5 kg and a flight time of 50 minutes. Many other drones, commercial or custom, may, of course, be used in place of the AURELIA® X4 drone.

The STU 116 of the embodiment shown is composed of a main body 116B that has a submersible portion 116P that holds a plurality of submersible sensors 128S, here, four submersible sensors 128S(1) through 128S(4). In this example, the four submersible sensors 128S(1) through 128S(4) are, respectively, a temperature sensor, a pH sensor, a turbidity sensor, and a TDS sensor. More or fewer submersible sensors 128S may be provided and/or submersible sensors of one or more differing types may be provided, if/as needed to suit a particular mission or type of mission. The submersible sensors may be part of an overall sensor system 128 that includes any onboard electronics 128E that may be needed to operate one or more of the submersible sensors 128S, including, but not limited to and as may be needed, one or more analog-to-digital converters (not shown), one or more signal conditioners (not shown; e.g., amplifier(s), filter(s), etc.), one or more signal generator(s) for any active sensor (e.g., turbidity sensor, TDS sensor, conductivity sensor, etc.) and/or one or more processors (not shown) and needed memory (not shown) for controlling operation of one or more of the submersible sensors. Those skilled in the art will readily understand the needs for any type of sensor system 128 provided and will be able to provide such a sensor system without undue experimentation.

In this example, the STU 116 also includes a sampling system 132 having a plurality of sample chambers 132C(1) through 132C(N) and one or more sample-chamber structures 132S, here, sample-chamber structure 132S(1) through 132S(N) to match the number of the sample chambers, that define the sample chambers. While this example shows a 1:1 correspondence between the sample chambers 132C(1) through 132C(N) and the sample-chamber structures 132S(1) through 132S(N), in other embodiments the ratio may be different. For example, a single sample-chamber structure (not shown) may define two or more, or all, of the sample chambers provided on a particular STU. The number, N, of the sample chambers 132C(1) through 132C(N) may be any integer number desired, for example, 2 through 24, or more. In this example, each sample chamber 132C(1) through 132C(N) has a corresponding inlet 132I(1) through 132I(N) for allowing water to flow into the corresponding sample chamber during sampling. As noted above, each inlet may be composed of a single opening or multiple openings in the corresponding sample-chamber structure 132S(1) through 132S(N).

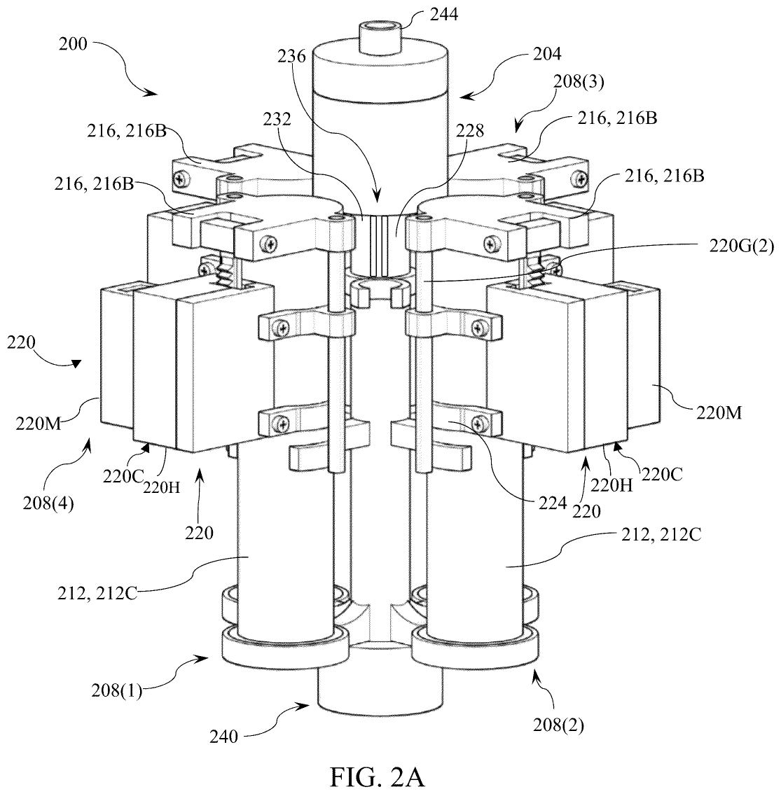

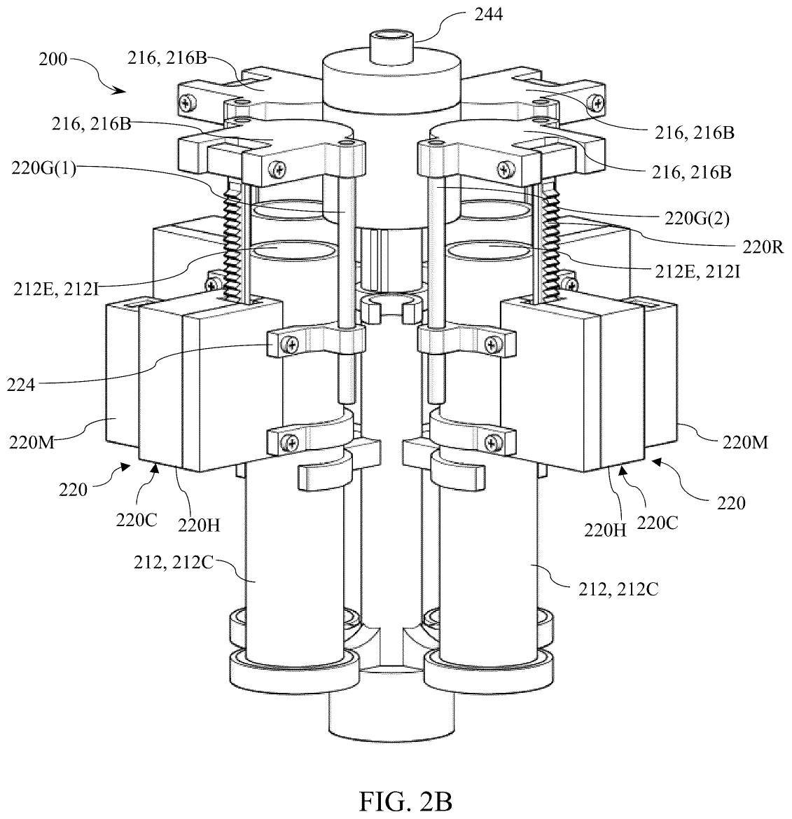

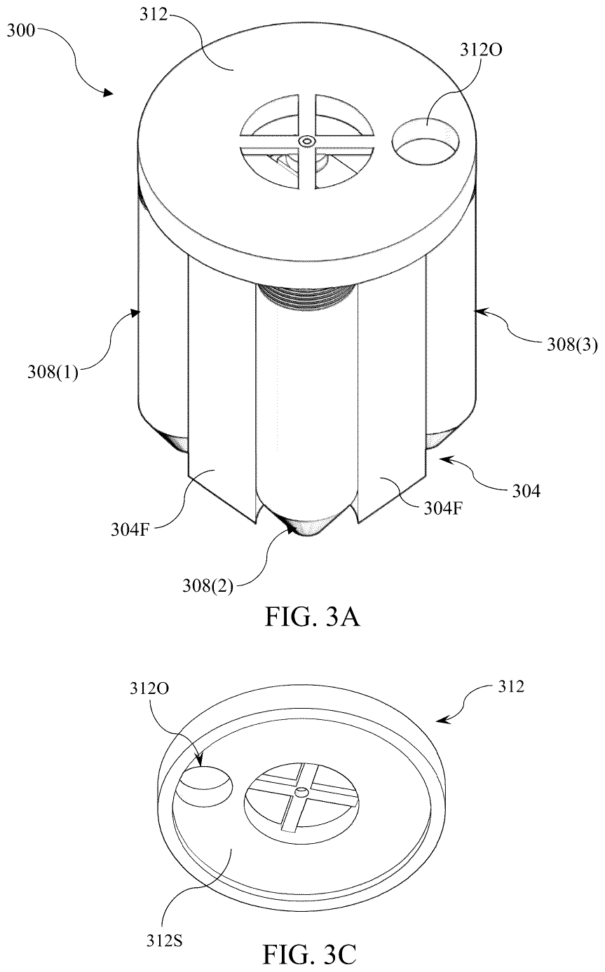

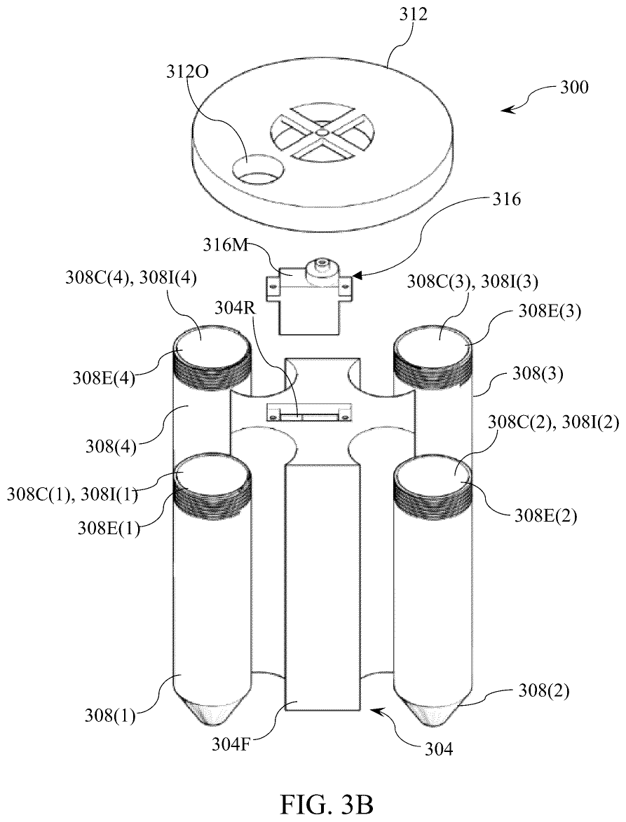

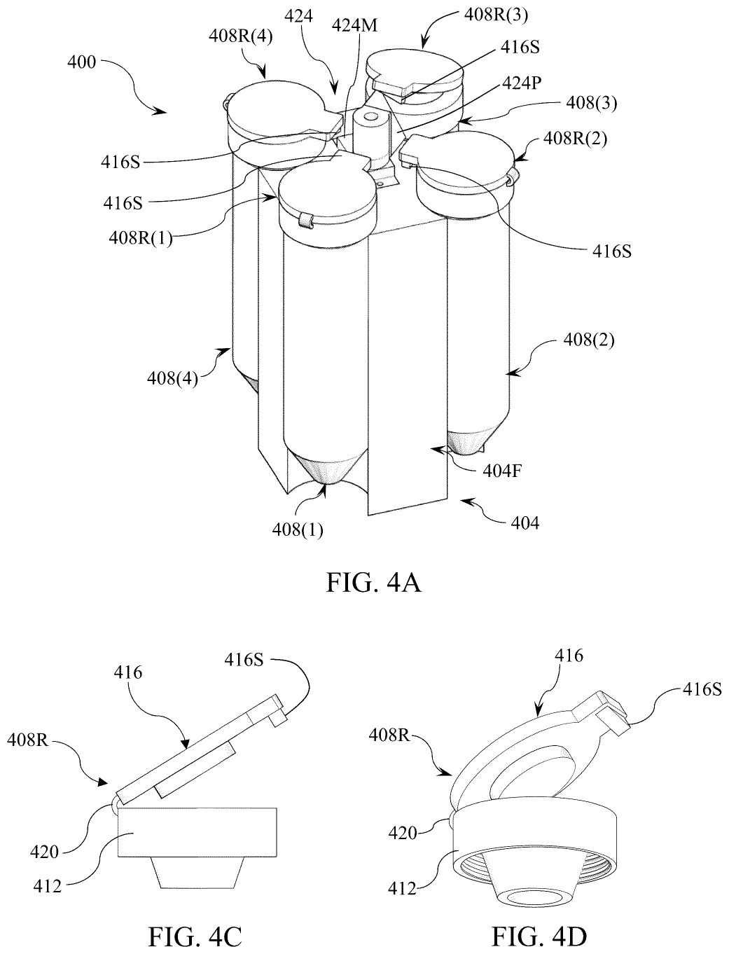

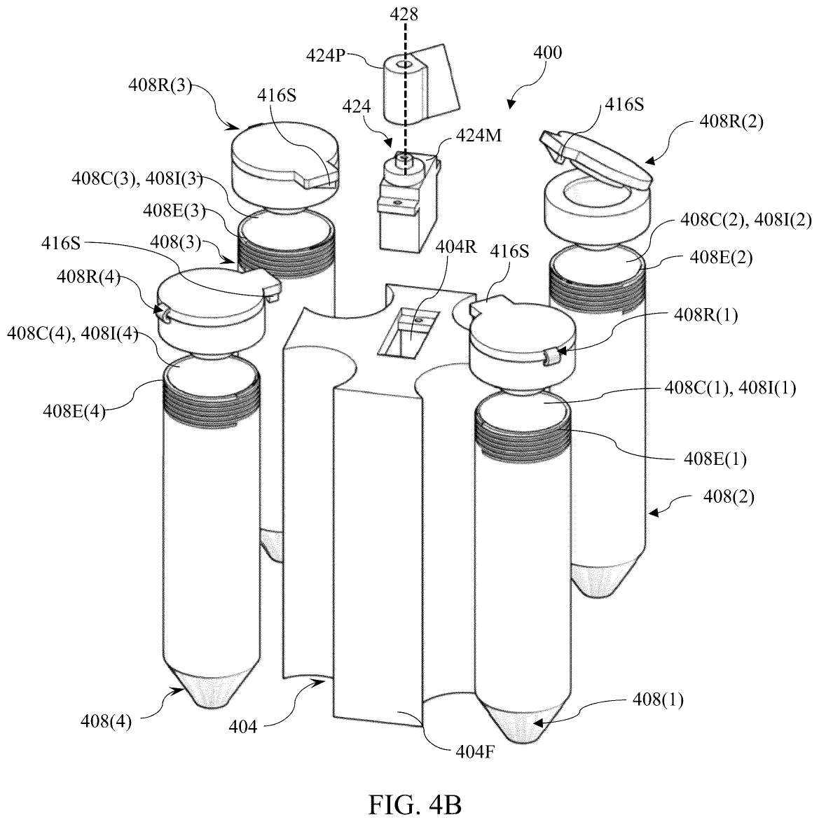

The sampling system 132 includes at least one sealing member 132M for sealing the inlets 1321(1) through 132I(N) to the sample chambers 132C(1) through 132C(N) at times other than when sampling is occurring. In some embodiments, each inlet 1321(1) through 132I(N) is sealed by a corresponding sealing member 132M(1) through 132M(N) that is unique to that inlet. FIGS. 2A-2B and 4A-4D show examples of STUs 200 and 400, respectively, that have a 1:1 ratio between the sealing members 216 and 416S the corresponding sample chambers 212C and 408C(1) through 408C(4). In some embodiments, a single sealing member seals all of the inlets 132I(1) through 132I(N), with an example of such appearing in FIGS. 3A-3C. In other embodiments that are not illustrated, a single sealing member 132M may seal two or more, but fewer than all, of the inlets 1321(1) through 132I(N). For example, some missions may require that two samples be acquired at each geographic location. In that case, N/2 sealing members 132M may be provided for N sample chambers 132C(1) through 132C(N), with each sealing member sealing two corresponding sample chambers such that actuating a single sealing member simultaneously opens two inlets 132I(1) through 132I(N). Many other possibilities exist, as those skilled in the art can readily envision.

In this embodiment, the sampling system 132 further includes at least one seal-actuation mechanism 132A for actuating each of the one or more sealing members 132M. As noted above in the General section, each seal-actuation mechanism can include, for example, a linear actuator (e.g., linear motor, pneumatic piston, hydraulic piston, etc.) or a rotational motor (e.g., a stepper motor, a servomotor, etc.), either alone or in combination with one or more other components, such as a gearbox and/or motion converter (e.g., a rack and pinion assembly, etc.). FIGS. 2A-2B illustrate an example STU 200 in which the sampling system has a seal-actuation mechanism 220 for each of the sealing members 216. FIGS. 4A-4D illustrate an example STU 400 in which the sampling system has a single seal-actuation mechanism 424 for moving each of the sealing members 416S of the multiple inlets 4081(1) through 408I(4). FIGS. 3A-3C illustrate an example STU 300 in which the sampling system has a single seal-actuation mechanism 316 for moving a single sealing member 312 that seals and unseals and unseals all of the inlets 308I(1) through 308I(4) in series.

The sampling system 132 may further include any onboard electronics 132E that may be needed to operate each of the seal-actuation mechanisms 132A, including, but not limited to and as may be needed, one or more digital-to-analog converters (not shown), one or more signal conditioners (not shown; e.g., amplifier(s), filter(s), etc.), one or more motor-control signal generator(s), and/or one or more processors (not shown) and needed memory (not shown) for controlling operation of each of the seal-actuation mechanisms. Those skilled in the art will readily understand the needs for any type of sampling system 132 provided and will be able to provide such a sampling system without undue experimentation.

The STU deployment system 104 may optionally include an imaging system 136 for acquiring images of water in the relevant body of water that is the target of a particular mission. In an example, imaging system 136 is distinct from any imaging system (not shown) that may be part of the UAV 120. The imaging system 136, if provided, may include one or more imaging devices, singly and collectively represented at element 136D. The imaging device(s) 136D may be of any type(s) desired or needed for a particular mission or particular type of mission. Examples of imaging devices that each imaging device 136D may be include, but are not limited to, a visible light camera, a near-infrared (NIR) camera, and a Lidar camera, among others. Those skilled in the art will be familiar with various types of imaging devices that may be desirable or needed to include on the STU deployment system 104 such that further explanation is not needed herein for those skilled in the art to be able to practice such aspects of the present disclosure without undue experimentation.

The STU deployment system 104 includes a data communications system 140 for transmitting sensor data collected by the sensor system 128 to one or more locations offboard of the STU deployment system, and, in some embodiments, any other relevant information about the status of the STU 116 and/or the UAV. The data communications system 140 may also be configured for receiving instructions/commands from offboard the STU deployment system 104 for controlling the sensor system 128 and/or for controlling the sampling system 132, depending on the configuration of the STU system 100. If the STU deployment system 104 includes an imaging system, the data communications system 140 may also transmit images captured thereby to one or more locations offboard of the STU deployment system. As noted above in the General section, the data communications system 140 may comprise any communications hardware 140H for the task, including, but not limited to LoRaWAN hardware, among others. Depending on the desired design of the STU deployment system 104, the communications hardware 140H may be located, for example, on the STU 116 or the UAV 120, or split between the STU and the UAV.

The STU deployment system 104 may include a positioning system 144 that allows the STU system 100 to determine the locations on a body of water where submersible-sensor data is acquired and/or water samples are acquired. Locational data from the positioning system 144 can be used for any one or more of a variety of purposes, such as mapping test results, planning testing locations, and determining a flight path, among others. In some embodiments, the positioning system 144 includes GPS hardware 144H. However, in other embodiments, the positioning system 144 may include another type of positioning hardware (not shown), such as a positioning system that uses terrestrial-based triangulation rather than satellite-based triangulation. The positioning system 144 may be located, for example, onboard the STU 116 or the UAV 120.

In some embodiments, the STU deployment system 104 may include a navigation controller 148 that can be configured to provide navigation functionality to the mobile sensing robot. As alluded to above in the General section, the STU deployment system 104 can be provided with any one or more desired levels of navigation functionality, from fully human guided, to semi-autonomous, to fully autonomous, and the navigation controller 148 is configured to provide the requisite functionality(ies) for enabling these functionalities. In this connection, the navigation controller 148 may include any suitable hardware 148H and software 148S needed to enable these functionalities. Consequently, the navigation controller 148 may be in operative communication, as needed or as available, with any one or more of the UAV 120, the imaging systems 136, and the communications systems 140, among other things. Further details and examples of navigation are described in the General section above. Like other hardware aboard the STU deployment system 104, the hardware 148H provided may be dedicated to the navigation controller 148 or it may be shared with one or more other systems aboard the STU deployment system, such as the sensor system 128, the sampling system 132, the imaging system 136 and the communications system 140, as may be available or make practical sense.

In some embodiments, the STU deployment system 104 includes a control system 152 that may act as a central controller aboard the STU deployment system to coordinate operations of any two or more other components aboard the STU deployment system, such as the UAV 120, the navigation controller 148, the sensor system 128, the sampling system 132, the imaging system 136, and the communications system 140. The control system 152 may include hardware 152H, for executing software 152S, that is separate from or shared with one or more other components aboard the STU deployment system, such as the navigation controller 148, the sensor system 128, the sampling system 132, the imaging system 136 and the communications system 140, as may be available or make practical sense.

In the illustrated embodiment, the STU system 100 includes an external interface system 156 that allows a human user to perform any one or more of the following example tasks: view sensor data that the sensor system 128 acquires, view images that the imaging system 136 acquires, view a map containing the body of water at issue, control movement of the STU deployment system 104, issue higher-level navigation commands to the STU deployment system, control operation of the sensor system, control operation of the sampling system 132, and control operation of the imaging system, among other things. In this example, the external interface system 156 includes STU software 156S that provides the requisite functionalities for enabling any one or more of the aforementioned tasks and/or other related tasks, such as storing sensor data, images, and/or other data concerning the STU system 100, among other things. Each functionality provided by the STU software 156S is encoded in computer-executable instructions (not shown) in one or more algorithms (not shown) as is customary and well-known in the art. The STU software 156S is stored in any suitable memory 156M and is executed by any suitable non-memory hardware 156H. It should be appreciated that while each of the memory 156M and the hardware 156H is shown as a single block, this is done only for convenience. In actual deployments, the memory 156M may be distributed among multiple network-connected devices, including, but not limited to multiple servers (not shown) in single server farm or in multiple server farms, among others. The non-memory hardware 156H may be distributed in a similar manner. Fundamentally, there are no physical and locational limitations on either the memory 156M or the non-memory hardware 156H.

The external interface system 156 may include one or more STU consoles 156C, each of which may be any suitable device, such as a laptop computer, a desktop computer, a cloud-connected device, a tablet computer, augmented reality headset, a dedicated console, etc. The term “STU console” is used for convenience and may be considered a combination of the physical device in combination with software that enables any one or more functionalities of the STU system 100, such as the STU software 156S. It is noted that while in some embodiments much of the computing and control capabilities of STU deployment system of the present disclosure, such as the STU deployment system 104, may be located aboard the STU deployment system, depending on the speed of the communications between the external interface system 156 and the STU deployment system, at least some of the computing and control capabilities may be provided in the external interface system.

Not shown, but which will be present in non-tethered embodiments, are one or more power sources, such as one or more batteries (e.g., rechargeable battery(ies)), aboard the STU deployment system 104 for powering one or more of the various components aboard the STU deployment system.

In some embodiments, the STU 116 may include an optional base 116OB that may remain with the tether 124 and allow the main body 116B to be swapped out for another main body (not shown). For example, the swapped-in main body may be different from the main body 116B shown in any one or more of a variety of ways, such as, but not limited to, having a different number of sample chambers 132C(1) through 132C(N), having fresh (i.e., unfilled) sample chambers, and having a different set of submersible sensors 128S(1) through 128S(4), among others. In some embodiments, any one or more of the submersible sensors 128S(1) through 128S(4) and/or other submersible sensors may be located on the optional base 116OB. For example, all of the submersible sensors 128S(1) through 128(4) may be located onboard the optional base 116OB, with the main body 116B generally containing only the sampling system 132. Many variations are possible when providing the optional base 116OB.

The electronics 132E, such as electronics that control and/or power the operation of the sampling system 132, and/or any of the electronics 128E, such as electronics that control and/or power the operation of the sensor system 128, that may be present onboard the STU 116 may be powered by the one or more batteries mentioned above. In some embodiments, one or more of the one or more batteries may be rechargeable. If so, the STU deployment system 104 may optionally include a charging dock 160, separate from the STU 116, that the STU can be engaged with for charging the onboard battery(ies). For example, the STU 116 may be configured to be docked on the charging dock 160 at most times other than when it is deployed for use. To facilitate docking, the STU 116 and the charging dock 160 may have respective sets of electrical contacts 164 that electrically contact one another while the STU is docked. Example electrical contacts include, but are not limited to, pogo pins, stationary flat contacts, and cantilevered, spring-loaded contacts, among many others. Fundamentally, there is/are no limitations on the type(s) of electrical contacts that can be used for the respective sets of electrical contacts 164.

In some embodiments in which some or all of the electronics for operating the sampling system 132 and/or the sensor system 128 is located onboard the STU 116, the STU may optionally include a magnetic switch 168 that is located within a watertight compartment 172 of the STU and that allows a user to turn the STU on and off. In this example, the magnetic switch 168 is actuated by a magnet 176 located externally to the STU 116, such as a magnet integrated into a handheld fob or provided in another form. When the user wants to turn the STU 116 on or off, the user (not shown) positions the magnet 176, on the outside of the STU, close to the STU and the magnetic switch 168 so as to trigger the magnetic switch located inside the watertight compartment 172 to close or open, as the case may be. In this connection, the main body 116B may include one or more indicia, such as writing and/or one or more icons, on its outside surface at the location of the magnetic switch 168 on the inside so as to alert the user where to position the external magnet 176. Although not shown, the STU 116 may optionally include an indicator light (e.g., LED) that indicates the off and on states of the STU. Providing the magnetic switch 168 reduces the number of potential water-leak points between the watertight compartment 172 and water (not shown) that surrounds the STU during use of the STU. In some embodiments, the STU 116 may have a single large watertight compartment 172 that houses all electronic components aboard the STU. However, those skilled in the art will readily appreciate that more than one watertight compartment may be provided, depending on the design at issue. In some embodiments, some or all of the control systems and/or some or all of the batteries may be located aboard the UAV 120 or other aerial vehicle, with control signals and/or power provided via the tether 124.

In some embodiments, the STU deployment system 104 may optionally include a disconnect device 180 located between the STU 116 and the UAV 120. When provided, the disconnect device 180 can be located at any suitable location, for example, within the tether 124, i.e., so that a length of tether is located on each side of the disconnect device, or at the location where the tether joins the base 116OB or the main body 116B of the STU 116. In some instantiations, the disconnect device 180 may be configured to cause the STU 116 to disconnect from the UAV 120 when the tensile force on the tether 124 exceeds a predetermined limit. This disconnect feature can be beneficial, for example, when the STU 116 has been deployed and gets snagged or caught in a submerged object, plant growth, or other matter, as it allows the UAV 120 to be safely returned when the STU needs to be sacrificed. In some instantiations, the disconnect device 180 may be configured to release the STU 116 from a remote location, for example, from any one or more of the STU consoles 156C, among other locations. In some instantiations, the disconnect device 180 may be configured to have both a force-based release and a remotely triggered release. In some instantiations, including in any of the foregoing instantiations, the disconnect device 180 may include a manual release that a user can actuate while handling the disconnect device. Such manual release, for example, allows a user to swap out one instantiation of the STU 116 for another instantiation of the STU. As those skilled in the art will readily appreciate, known release mechanisms can be used for each of the force-based release, the remotely triggered release, and the local manual release.

In some embodiments, each of the sample chamber structures 132S(1) through 132S(N) may optionally be removably secured to the main body 116B of the STU 116. For example, each of the sample chamber structures 132S(1) through 132S(N) may be removably secured to the main body 116B by a friction fit with a corresponding holder (not shown), by a magnetic connection, by a mechanical latching system, or by a threaded connection, among many others, and any meaningful combination thereof.

Any aspect of the example sampling system 100 of FIG. 1 not specifically addressed may be the same or similar to the corresponding aspect discussed above in the General section.

FIGS. 2A and 2B illustrate an example STU 200 made in accordance with aspects of the present disclosure. In this example, the STU 200 includes a main body 204 to which are secured four sample-chamber assemblies 208(1) through 208(4) that are identical to one another. Each sample-chamber assembly 208(1) through 208(4) includes a sample-chamber structure 212 defining a sample chamber 212C (only some visible), a sealing member 216 that removably seals the sample chamber, a seal-actuation mechanism 220 (only some visible) for moving the sealing member into and out of sealing engagement with the sample-chamber structure, and a support frame 224 (only some visible) that supports all of these components.

In this example, the sample-chamber structure 212 is composed of a glass tube having an open end 212E (FIG. 2B) that provides an inlet 212I to the sample chamber 212C. The sealing member 216 of this example has a rigid backing 216B to which is affixed a seal (not shown, but on the underside of the rigid backing) that engages the open end 212E of the sample-chamber structure 212 when the sealing member is in its sealing position. The seal may be made of any suable material, such as a compliant elastomer, among others.

As can be seen by comparing the positions of the sealing members 216 as between FIGS. 2A and 2B, the seal-actuation mechanism 220 moves the sealing member away from and toward the open end 212E of the sample-chamber structure 212 so as to effect unsealing and sealing, respectively. In this connection, the seal-actuation mechanism 220 includes a rotational motor 220M (only some labeled), motion converter 220C (only some labeled) that includes a rack 220R (only one labeled) and corresponding pinion (not seen, but inside a housing 220H), and a pair of linear guides 220G(1) and 220G (2) (only some labeled). As can be appreciated, the rotational motor 220M drives the pinion, which engages the rack 212R to move the sealing member 216 relative to the sample-chamber structure 212.

In this embodiment and referring to FIG. 2A, the STU 200 also includes a temperature sensor 228, a pH sensor 232, a TDS sensor 236, and a turbidity sensor 240, each of which may be of any known suitable type. In this example, the entirety of the STU 200 may be considered the submersible portion, as the entire STU may be submerged beneath the surface of the water being tested. The STU 200 shown in FIGS. 2A and 2B is secured to a tether 244, which includes, hidden inside, both electrical power wires and sensor data cables.

FIGS. 3A and 3B illustrate another example STU 300 made in accordance with aspects of the present disclosure. In this example, the STU 300 includes a main body 304 and four sample-chamber structures 308(1) through 308(4), which in this example are embodied as conventional centrifuge tubes that are removably held by the main body by a snap-in-place fit. As seen particularly in FIG. 3B, each of the sample-chamber structures 308(1) through 308(4) defines a corresponding sample chamber 308C(1) through 308C(4) and has an open end 308E(1) through 308E(4) that provides an inlet 308I(1) through 308I(4) to the corresponding sample chamber.

The STU 300 includes a single sealing member 312 that includes an opening 3120 that, when positioned over any one of the inlets 308I(1) through 308I(4) (FIG. 3B) allows water in which the STU 300 is submerged to flow into the corresponding sample chamber 308C(1) through 308C(4). Oppositely, when the opening 3120 is positioned between any two adjacent ones of the sample-chamber structure 308(1) through 308(4), the sealing member 312 seals all four of the inlets 3081(1) through 308I(4). As seen in FIG. 3C, the underside of the sealing member 312 has a seal 312S that effects the seal with the corresponding sample-chamber structure 308(1) through 308(4). In some embodiments, the seal 312S is composed of a compliant material, such as an elastomeric material, among others.

To move the opening 3120 relative to the inlets 3081(1) through 308I(4), the STU includes a seal-actuation mechanism 316, which in this example is simply an electric rotational motor 316M, such as a stepper motor that can readily keep track of the location of the opening relative to the inlets. In this example, the motor 316M is mounted within a motor receptacle 304R formed in the main body 304. The main body 304 may be made of any one or more suitable materials, especially one or more relatively lightweight materials to minimize the lifting burden of any UAV (not shown) that may carry the STU. Although not seen in FIGS. 3A and 3B, the inside of the main body 304 may be hollow to minimize its weight and to provide space for any necessary/desired electronics, battery(ies), sensor drivers, etc. Other electronics are not shown in FIGS. 3A and 3B, nor are any submersible sensors, like the submersible sensors 228, 232, 236, and 240 shown on the STU 200 of FIGS. 2A and 2B. However, any one or more of these things may be provided as needed/desired. Regarding submersible sensors, one or more can be mounted on any of the faces 304F (only some visible and labeled) of the main body 304 and/or on the bottom of the main body. Alternatively, if a base (not shown) to which the STU 300 shown may be mounted is provided, such electronics and/or submersible sensor(s) can be provided in or on that base. Those skilled in the art will readily appreciate that many variation of the general theme of the STU 300 of FIGS. 3A through 3C can be made using only ordinary skill in the art and this disclosure as a guide.

FIGS. 4A and 4B illustrate a further example STU 400 made in accordance with aspects of the present disclosure. In this example, the STU 400 includes a main body 404 and four sample-chamber structures 408(1) through 408(4), which in this example are embodied as conventional centrifuge tubes that are removably held by the main body by a snap-in-place fit and include corresponding threaded removable caps 408R(1) through 408R(4). As seen particularly in FIG. 4B, each of the sample-chamber structures 408(1) through 408(4) defines a corresponding sample chamber 408C(1) through 408C(4) and has an open end 408E(1) through 408E(4) that provides an inlet 408I(1) through 408I(4) to the corresponding sample chamber.

As seen in each of FIGS. 4C and 4D, each threaded cap 408R includes a base 412 and a sealing member 416 that is hingedly secured to the base via a hinge 420. Each hinge 420 is designed and configured to bias the sealing member 416 into a sealing position that provides a watertight seal for the corresponding sample chamber 408C(1)-(4) (FIG. 4B). Referring again to FIGS. 4C and 4D, each sealing member 416 includes a strike member 416S that cooperates with a seal-actuation mechanism 424 (FIGS. 4A and 4B) as described below.

Referring again to FIGS. 4A and 4B, the seal-actuation mechanism 424 includes an electric rotational motor 424M that, in this example, directly drives a pusher 424P that is rotatable 360° about a rotational axis 428 of the motor. The motor 424M may be, for example, either a stepper motor or a servomotor. The pusher 424P is designed and configured to engage each strike member 416S of each sealing member 416 so as to pivot that strike member and hold it in an open position as water in which the STU 400 is submerged flows into the corresponding sample chamber 418C(1) through 408C(4) (FIG. 4B). Once the motor 424M moves the pusher 424P past the strike member 416S that it is presently engaging, the corresponding hinge 420 returns the corresponding sealing member 416 to its sealing position. Although not shown, the pusher 424P may be complemented by a closure member that the motor 424M rotates in unison with the pusher in a trailing manner and that forces the sealing member 416 into firm sealing engagement with the corresponding base 420 to ensure a watertight seal.

In this example, the motor 424M is mounted within a motor receptacle 404R formed in the main body 404. The main body 404 may be made of any one or more suitable materials, especially one or more relatively lightweight materials to minimize the lifting burden of any UAV (not shown) that may carry the STU. Although not seen in FIGS. 4A and 4B, the inside of the main body 404 may be hollow to minimize its weight and to provide space for any necessary/desired electronics, battery(ies), sensor drivers, etc. Other electronics are not shown in FIGS. 4A and 4B, nor are any submersible sensors, like the submersible sensors 228, 232, 236, and 240 shown on the STU 200 of FIGS. 2A and 2B. However, any one or more of these things may be provided as needed/desired. Regarding submersible sensors, one or more can be mounted on any of the faces 404F of the main body 404 and/or on the bottom of the main body. Alternatively, if a base (not shown) to which the STU 400 shown may be mounted is provided, such electronics and/or submersible sensor(s) can be provided in or on that base. Those skilled in the art will readily appreciate that many variations of the general theme of the STU 400 of FIGS. 4A through 4D can be made using only ordinary skill in the art and this disclosure as a guide.

### Example Implementation

With the foregoing in mind, this section describes some example implementations of the above-described STU systems and components thereof. These examples are intended to be illustrative and nonlimiting. It is noted that various commercial off-the-shelf (COTS) devices, such as microcontrollers, sensors, processors, LoRaWAN radios, GPS, etc., are used in the following descriptions. Those skilled in the art will readily understand how to correlate such devices to the STU systems described above based on their functionalities. Any trademarks used in the following descriptions are intellectual property of their respective owners.

This section presents components of an autonomous STU system that seamlessly integrated water sensors, Internet of Drones (IoD), and LoRaWAN communication for instantaneous water quality monitoring and data transmission. Additionally, it incorporated water sample collectors for laboratory analysis and leveraged multispectral imaging along with a Geographic Information System (GIS) for detecting and mapping of cyanobacteria distribution. Multispectral imaging yielded valuable information regarding harmful algal bloom (HAB) distribution on the surface of a body of water, while GIS mapping offered a comprehensive understanding of HAB distribution. It aided in pinpointing hotspots and enabled dynamic modeling to delve deeper into the profile of these blooms. The STU system's integration empowered it to efficiently monitor various environmental parameters contributing to water quality, including temperature, pH, TDS, and turbidity (TBD). The system developed in this work used the activation-by-personalization (ABP) LoRaWAN protocol to wirelessly transmit water-quality parameters to a data server on the cloud for real-time data processing and analysis. Additionally, the STU system enhanced data resilience by taking backup onto a micro-SD card. The STU deployment system carried four automated water samplers, each capable of collecting 25 mL of samples. These four individual sampling channels offered flexibility in selecting a sampling volume from 25 mL to a combined total of 100 mL. The sampling strategy could be customized according to test needs. The open architecture of the sensor system facilitated seamless expansion to include one or more additional sensors, such as, but not limited to a depth sensor, a dissolved oxygen (O₂) sensor, a carbon dioxide (CO₂) sensor, nutrient sensors, a fluorometer, and heavy metal sensors, so as to enhance its monitoring capabilities.

### Architecture

This section provides a detailed overview of the embedded system design of the STU system. The system integrated sensors, automated water samplers, real-time wireless data transmission, micro-SD card backup storage, true-color and NIR cameras, and GIS mapping.

### Embedded Sensor System Design

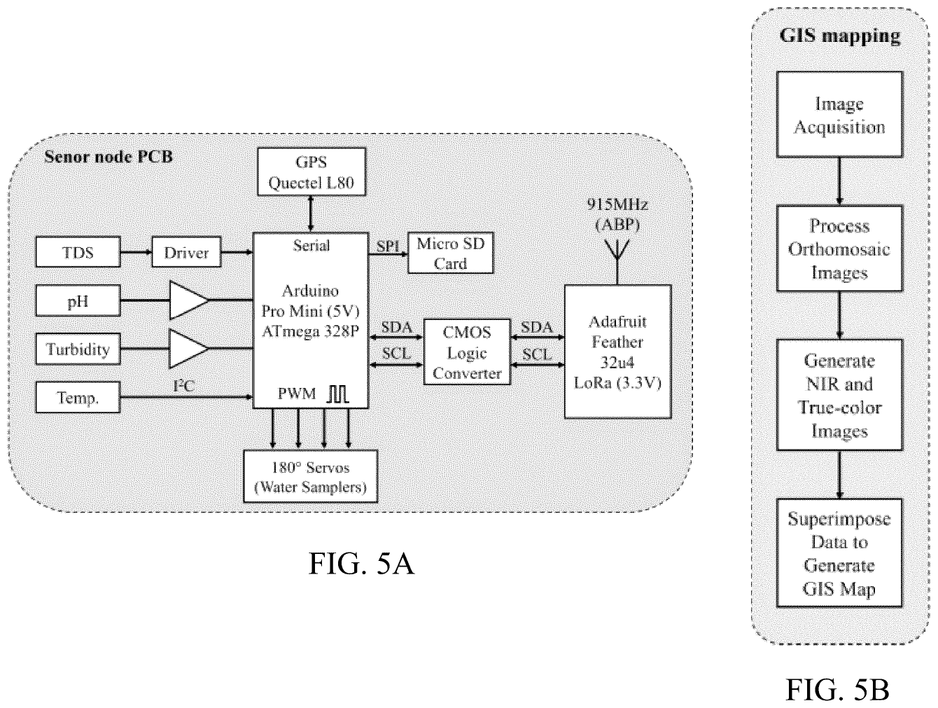

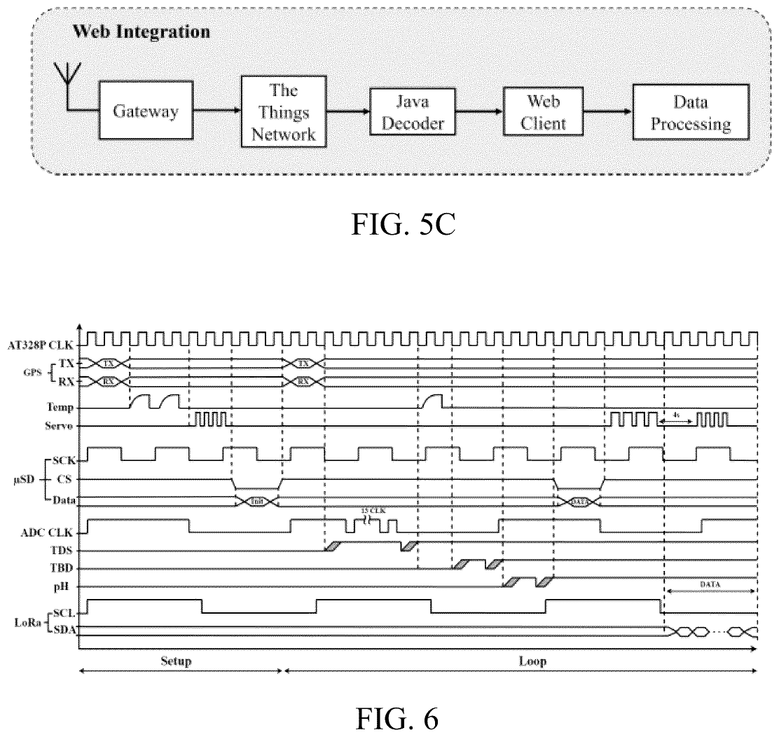

The main circuit board integrated key components such as sensor drivers, a Quectel L80 high-precision GPS module with circular error probability (CEP) less than 2.5 m, a micro-SD card module for local storage, an Arduino Pro Mini 5 V microcontroller, an Adafruit feather 32u4 RFM95 LoRa transceiver, and a bi-directional level shifter for inter-integrated circuit (I2C) communication between ATmega328P and ATmega32u4 microcontrollers, and sensors for measuring certain water quality parameters, including temperature, pH, TBD, and TDS. The adopted sensors were a GravityTDS sensor, a DFRobot turbidity sensor, a Hilitand pH (0 to 14) module, and a digital temperature sensor (DS18B20). TBD, pH, and TDS sensor outputs were read with a 10-bit analog-to-digital converter (ADC) of the ATmega328P microcontroller with a 5 V internal reference voltage, which provided digital values from 0 to 1023. Temperature sensor required a different protocol that is explained further below. FIGS. 5A-5C illustrate the integrated architecture for the STU system in terms of the sensor node printed circuit board (PCB), the GIS mapping, and the web integration.

Power supply: The required voltages in the circuit were ±3.0 V, +3.3 V, and +5 V. The sensor node circuit could be powered with battery voltages between 7 V and 25 V. An LM7805 positive voltage regulator provided a stable +5 V voltage. The 5 V voltage was then fed to an AMS1117 voltage regulator to generate a +3.3 V voltage. An ME6206 regulator generated a stable +3.0 V from the +5 V. A switching charge pump voltage inverter (TPS60400) generated a −3.0 V potential from the +3.0 V provided in the previous step. Each regulator had decoupling capacitors for high-frequency noise attenuation and voltage stabilization.

Temperature: The digital DS18B20 temperature sensor was utilized with a 1-wire protocol. The sensor was powered via +5 V and the data pin was pulled up with a 4.7 k ohm resistor.

pH: The pH probe was of laboratory grade. It was connected to the main board via a Bayonet Neill-Concelman (BNC) connector. The signal conversion board was powered by +5 V and the PO pin was used as the sensor output. One of as set of potentiometers (next to the BNC connector) was utilized for the main calibration, and the other one was for a digital output threshold adjustment. To calibrate the pH module, the BNC output was shorted, the main potentiometer was adjusted to produce a +2.5 V stable output, and then the probe was calibrated.

Turbidity: The DFRobot turbidity sensor was powered by +5 V and operated based on a light attenuation method, hence an inverse relationship between turbidity and output voltage. The phototransistor detector signal was fed to an analog buffer and the output had a DC voltage range between 0 V and 5 V. Each turbidity value was calculated in Nephelometric Turbidity Units (NTU). The calibration for turbidity was performed for concentrations that could be approximated by a linear response and the calibration relationship between the sensor output voltage and turbidity value is shown in Equation 1, below.

$$T B D ( N T U ) = - 1079.74 V + 4437.95 \qquad ( 1 )$$

TDS: The integrated GravityTDS driver and signal generator could measure dissolved solids between 0 and 1000 ppm. The TDS driver was powered by a ±3.0 V dual-polarity voltage source. The excitation signal was generated using a CD4060 14-stage binary ripple counter. The clock frequency for the oscillator was set to 40 kHz by Rt and Ct according to Equation 2, below. The alternating current (AC) signal prevented the probe electrodes from polarization and prolonged their life.

$$f = {( 2.5 R t C t )}^{- 1} \qquad ( 2 )$$

A 2.5 kHz pulse width modulation (PWM) pulse signal was generated with a 50% duty cycle which was fed to an inverting amplifier (gain=−0.068). The TDS output transitioned from low to high after 16 clock pulses. A full-wave precision rectifier was utilized to produce a positive voltage reading for either polarity of the input.

Micro-SD: A micro-SD card circuit was powered by +3.3 V and consisted of a socket and a four-channel buffer gate used as a level shifter. The logic level shifter was used for converting the logic level for the Micro-SD card. The Arduino microcontroller and the micro-SD communicated through the serial peripheral interface (SPI).

GPS: The L80 Quectel GPS module was powered by the +3.3 V voltage source. The GPS required UART communication. The 3D position fix, which implies the satellite connection required a minimum of four visible satellites and took up to 35 seconds while the information update rate was 1 Hz. The backup pin (V_BCKP) was connected to a second +3.3 V power supply voltage to maintain the satellite connection if the ATmega328P microcontroller was reset or turned off.

CMOS bi-directional logic level converter: Due to the difference in logic levels between microcontrollers ATmega32u4 (3.3 V logic level) and ATmega328P (5 V logic level), a bi-directional logic level converter was needed for their I2C communication.

A 2-layer PCB had an FR-4 material with a thickness of 1.6 mm. All devices were surface-mounted devices (SMDs). An 8.4 V 2200 mAh LiPo battery was used to power the whole circuit. The Adafruit feather LoRa was also powered from the 5 V voltage source via its USB power pin. Data-carrying traces matched with the SMD IC pads and were 1.0 mm or 0.45 mm.

The sensor node could continuously operate for 2 hours and 55 minutes using a 2200 mAh battery in active mode. The board could remain in standby mode for about 20 hours and 22 minutes.

Upon sensing water presence via the TDS sensor (see below), the sensor node entered an active mode, and water sample collection and wireless water quality data transmission began while the UAV was hovering for two minutes over each testing/sampling location. After sample collection and data transmission were completed, the sensor node entered a standby mode as soon as the apparatus was lifted out of the body of water.

### LoRaWAN Protocol

A LoRaWAN gateway linked the end devices with a LoRaWAN network server in the cloud. It forwarded the information to the network server after receiving it from the end nodes. A portable power station was utilized to power a multi-connect LoRaWAN gateway with a Wi-Fi Internet connection. This gateway connected to a 5 GHz Wi-Fi hotspot with WPA2-Personal security. The LoRa connection used the Feather 32u4 LoRa end node at the frequency plan of 902 MHz to 928 MHz registered on The Things Network (TTN) via the ABP protocol. Session keys (AppSKey and NwkSKey) and DevAddr were stored in non-volatile memory of the ATmega32u4 microcontroller, and the TTN frame counter reset upon the first uplink. ABP was chosen for instant transmission and rapid uplink; ABP was faster than over-the-air activation (OTAA) protocol, which required an extra 60 seconds to perform a join procedure with the LoRaWAN network. To enhance security measures, the session keys were initially configured manually for both the end node and the application at the onset of each sampling session. However, in scenarios where data security was not a critical consideration, it was possible to establish and maintain the session keys unchanged throughout the STU system's operation. This adaptability proved particularly beneficial for applications seeking automated functionality, as it alleviated the need for frequent key modifications. The UAV sensor node transmitted data every 15 seconds at a 20 dBm transmission power, reaching an 800 m transmission distance. The data transmission packet contained 16 bytes, among which 8-byte was for sensor data, and the other 8-byte was for GPS information. To improve data payload size and reliability, each 16-bit integer sensor data was split into two bytes: high and low, enhancing precision and transmission range. GPS coordinates, including latitude and longitude, used 4 bytes each, preserving six decimal digits after the decimal point. The transmitted byte array was structured to convey specific sensor data in a well-defined order. The initial two bytes represented temperature data, the subsequent two bytes signified TDS data, the following two bytes encapsulated Turbidity data, the ensuing two bytes encapsulated pH data, and the final eight bytes encompassed GPS information.

### Automated Electromechanical Water Samplers

The automated water sampler was both pumpless and waterproof, achieved by integrating four 9-gram mini servo motors into acrylic container tubes and is shown as the STU 200 of FIG. 2. Each servo motor was controlled by the ATmega328P controller. Waterproof silicon glue was applied to servo containers and connectors. This system allowed for sampling volumes of 25 mL, 50 mL, 75 mL, and 100 mL based on water HAB content and test requirements. For sample collection, the four tubes were made of polymethyl methacrylate (PMMA) with 21 mm inner diameter and 25 mm outer diameter. All structures, excluding the PMMA tubes, were fabricated using 3D-printed Polylactic Acid (PLA) filaments with a 1.75 mm diameter and a 0.2 mm nozzle.

### Operation of the Sensing System

The following description delves into the sensing system's operation and real-time water quality monitoring. The sensing system employed the TDS sensor reading for accurate water presence detection.

### Sensor Node Program

This section discusses the ATmega328P and ATmega32u4 controller software programs. While the sensing was completely done by the ATmega328P microcontroller, the ATmega32u4 microcontroller was utilized for receiving and packing the data and transmitting it through the RFM95 module.

Upon initialization of both software programs, all modules connected to ATmega328P controller were powered and tested. The 1-wire communication protocol was initiated for the temperature sensor, followed by the GPS module configuration. Afterward, the ports used for sensor value collection were defined as inputs. To verify proper functionality, all servo motors were completely activated and then deactivated as part of an initial test. This operation opened and closed the samplers to ensure proper functionality and sealing.

Within the ATmega328P controller's loop, the first task was to ensure a stable 3D GPS fix by calling the GPS function. This ensures that the system had acquired a reliable 3D GPS satellite lock. Subsequently, the TDS sensor value was sampled, which undergoes the threshold check. The code promptly reacted when the TDS value surpasses 50 ppm. In such a case, it proceeded to acquire data from sensors, and GPS coordinates. Then information was stored and transmitted wirelessly. Concurrently, the system entered a water sampling mode, adjusting the servo motors to collect water samples as specified. The collected data was transmitted to the Feather 32u4 LoRa module via I2C and utilizing the LMIC library, and the program packaged the sensor readings into a byte array and transmitted them over the LoRaWAN network. The code structure handled events such as successful data transmissions and includes error-checking mechanisms.

FIG. 6 illustrates the timing sequence used in the embedded system, including signal details. The GPS module operated at a 9600-bps data rate, with the GPS LED indicating a 3D position fix when connected to more than 5 satellites. The temperature sensor initialization involved specific timing, with pulse durations between 60 us to 240 us. Servo motors used PWM pulses with a 20 ms period and 2 ms (for opening) and 1 ms (for closing) duty cycles for positioning. Other sensors (pH, TDS, TBD) employed a 10-bit ADC at 125 kHz with a 104 us conversion time and 15.4 k samples per second sampling rate. The micro-SD card's clock was set to 4 MHZ, and I2C communication with LoRa module and the Arduino microcontroller occurred at 100 kHz SCL frequency.

### Water Sensing and Real-Time Data Transmission

The water quality monitoring system employed four sensors to quantitatively measure water temperature, pH, TDS, and turbidity. All measurements were initiated exclusively when the complete sensor apparatus became fully immersed in the water body. The TDS sensor was used for dual purposes: water presence detection through its electrical conductivity measurement and water TDS value measurement. The TDS sensor offered a unique capability that made it highly effective for detecting the presence of water. Unlike the other three sensors (pH, temperature, and turbidity), upon submerging in water, the TDS probes experienced a change in electric charges due to the presence of dissolved solids, resulting in the generation of an electric current that was sensed by the full-wave precision rectifier. In the air and pure DI water, TDS readings were zero.

The water-sensing strategy was established based on a predetermined threshold for the TDS value. Pure water had no dissolved solids resulting in a 0 ppm TDS reading. TDS had a negligible reading of less than 10 ppm with small residual droplets. The threshold value for TDS was chosen such that it was within the measurement range of the device and implied a positive non-zero value. A value of 50 ppm was chosen for TDS since it is common in natural springs, lakes, and ponds. If the TDS value surpassed the predetermined threshold of 50 ppm, water presence was detected, triggering the STU to conduct water quality parameter measurements and water sample collection accordingly.

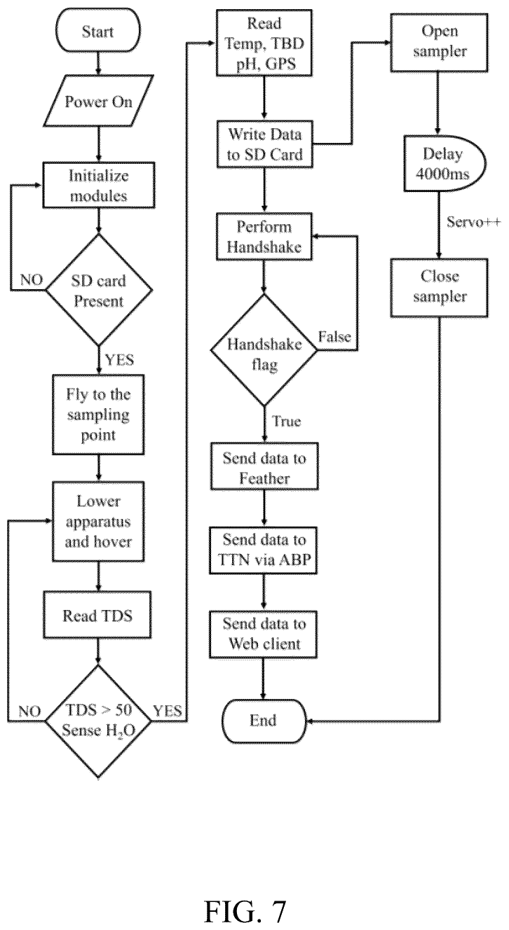

The approximate sampling depth profile could be calculated concerning the altitude of the UAV. This depth was calculable via subtracting the current UAV altitude from the combined length of the apparatus and tether (2.4 m). The connecting tether was a 12 AWG 12-conductor. FIG. 7 illustrates the sampling performed by this instantiation of an STU system. Initially, the battery was connected to the PCB, and the modules were checked. If the micro-SD card was in the socket, the system was activated. The UAV was then flown to the testing/sampling location of interest. After lowering the apparatus into the water body, and the TDS measurement exceeded 50 ppm, several simultaneous processes took place: reading the sensor values, storing data into a backup Micro-SD card, transmitting data wirelessly to the cloud, and collecting water samples. The Servo index determined the sealing member of the corresponding sample chamber. The STU remained in the water for 2 minutes to allow the sensors to stabilize. Meanwhile, the STU started to transmit data to the LoRa gateway.

There was a bi-directional handshake process between the ATmega328P and ATmega32u4 microcontrollers for synchronization, flow control, error handling, and device identification. If the two microcontrollers were synchronized, the Handshake flag was set to true for both, and it initiated the data transfer from ATmega328P microcontroller to ATmega32u4 microcontroller. When the data index equaled 6, implying full data transfer, the feather 32u4 LoRaWAN module began to transmit the data wirelessly through the LoRaWAN. The uplink containing water quality parameters was then received and processed by the LoRaWAN gateway in real time.

### Signal and Data Processing

The data processing was divided into cloud-based and on-chip processing. The Arduino microcontroller software code (on-chip processing) featured robust data processing for various sensors in a water sampling system. All sensors constantly generated readings that were transmitted to the cloud. The TDS sensor software code used a median filter for stability and compensated for the difference between the measured temperature and 25° C. pH readings underwent on-chip averaging for accuracy. Turbidity readings were calculated using a calibration curve. GPS data was processed for latitude and longitude. The data was then encoded into a LoRaWAN packet and transmitted. The gateway then received this LoRaWAN packet. JavaScript code decoded a LoRaWAN packet, specifically for port 7. It extracted temperature, TDS, turbidity, pH, latitude, and longitude readings. The code used byte slicing to extract specific ranges of bytes from the raw byte array. Additionally, there was a check for signed integers to handle values greater than or equal to 32768, ensuring correct decoding of sensor values. Cloud data processing involved converting received bytes to integer values and storing them. The stored data was then processed to calculate the average and standard deviation.

### Field Test and Study Site

This experiment section emphasizes the STU system testing and validation. Samples and real-time data were collected from 8 testing/sampling locations from the selected locations of a study site. The automated water samplers were washed and rinsed with ultra-pure DI water twice before each sampling mission to avoid cross-contamination. The entire sensing and sample collection process for all 8 locations took 45 minutes, encompassing water sample collection, water parameters measurement, wireless data transmission, flight between the selected testing/sampling locations and the launch station, sample tube preparation and cleaning, etc. At each testing/sensing location, the UAV hovered for only 2 minutes to complete the sensing and water sample collection. An onboard multispectral imaging camera surveyed the terrain to capture NIR imagery and facilitate precise GIS mapping before water parameters measurement and water sampling. Initially, the onboard multispectral imaging camera surveyed the terrain to capture NIR imagery and facilitate precise GIS mapping, and the main sample collection was performed shortly after the imaging.

### Study Site

This section focuses on a field test conducted at a pond that is a eutrophic body of water hosting large blooms of algae. The body of water is home to a variety of fish including Largemouth bass, Northern pike, Brown bullhead, Yellow perch, and panfish.

The pond covers an expansive area of approximately 1.9 km² and is surrounded by an extensive landscape encompassing 1.6 km² of swamps, marshes, and woods. This pond has been classified as a nutrient-impaired lake. Notably, it exhibits elevated levels of phosphorus and chlorophyll-a, while displaying low Secchi transparency during the spring and summer months, placing it in the hypereutrophic to eutrophic category according to the relevant standards. In the spring, macrophytes flourish, yet as the summer sets in, cyanobacteria blooms occur, triggering oxygen depletion and subsequent fish fatalities, primarily attributed to the oxygen deficiency stemming from the decay of these blooms.

### UAV Setup

This section is dedicated to the UAVs utilized in this project. The UAV was an Aurelia X4 drone that has two 10,000 mAh LiPo batteries for 50 minutes of flight time and a 1.5 kg payload capacity. A 3D-printed frame mounted the sampling PCB to the UAV. A LoRa antenna, the LiPo battery, and the 12-connector cable tether with the safety latch were secured to the UAV frame. The UAV was manually controlled via a Taranis Q X7 remote, hovering for only 2 minutes at each location to stabilize sensor readings. The swing effect occurred when the aircraft suddenly came to a full stop before landing. The effect took place due to the inertia of the apparatus according to Newton's first law of motion (inertia). This effect, however, was minimal and manageable at horizontal speeds≤1 m/s. Altitude and speed were recorded using an Ardupilot mission planner. A Mavic 3M imaging system captured 20 MP true color and 5 MP NIR images from the study site.

At each testing/sensing location, the STU was submerged in the water at a depth of 1 m. As noted the STU deployment system remained in the position shown 2 minutes. The safety latch was a 12-pin connector. If the force applied to the safety latch exceeds 19.6 N while the STU was deployed, the connector would disengage, safeguarding the UAV by releasing the STU. The airflow generated by the propellers of a UAV did not affect NIR imaging of algae distribution. NIR imaging was used for monitoring the water surface over a wide area, and this sensing was performed when the UAV was at a much higher altitude and was away from the sampling locations.

### Multispectral Image Acquisition and GIS Mapping

Images were captured from a radiometric calibration target that were used automatically by the Pix4Dfields program, along with the ambient light sensor information captured by the Mavic 3 UAV to generate multispectral imagery with accurate radiometric information. The drone featured RTK-grade GPS using the Vermont state NTRIP/VRS network for GPS corrections. It provided geotags of images with a horizontal accuracy of 3 cm to 8 cm.

Once the images were processed, they were exported from a Pix4Dfields software program as both true-color and multispectral files and brought into an ESRI ArcGIS Pro database. Next, a false composite image was created by applying a stretch to the NIR band. The stretch displayed values along a color ramp for a single band. An overall GIS mapping was created by superimposing water quality information, including temperature, pH, TDS, and turbidity, as attributes on top of the UAS imagery according to the testing/sampling location coordinates.

The system design provided two methods for temporal synchronization between image information and sample collection: satellite and cloud. Time information could either be obtained from satellite information or cloud time. In this study, temporal data was gathered from a web client and then synchronized by a 15-second adjustment to align with the transmission time, ensuring it matched the sampling time precisely.

### Results

This section discusses the results of laboratory analysis of collected water samples, multispectral imaging of the blue-green algae, and image processing techniques, as well as GIS mapping. Further details of the results can be found in the paper S. Faghir Hagh et al.: Autonomous UAV LoRaWAN System for Real-Time Monitoring of Harmful Algal Blooms (HABs) and Water Quality, IEEE SENSORS JOURNAL, VOL. 24, NO. 7, Apr. 1, 2024, pp. 11414-11424, which is incorporated herein by reference for all of its relevant disclosure.

### Laboratory Analysis and Study Site Parameters

Samples collected for laboratory determination of algal and cyanobacteria biomass (chlorophyll-a (Chl-a) and Phycocyanin (PC)), were transferred to 200 mL high-density polyethylene (HDPE) laboratory-grade containers and immediately put into a cooler with ice to reduce changes in samples. Samples were stored at a temperature of 4° C. until the measurements for 2 hours. To ensure accurate measurements, samples needed to be analyzed within 24 hours of collection from the water body. Phycocyanin, and chlorophyll-a, were measured with an Aqua TROLL 500 multiparameter sonde (In-Situ Inc., Fort Collins, CO, USA). PC levels have been reported using relative fluorescence units (RFU). Temperature and pH showed low variations across locations. PC increased by 7.77 RFU from the 2ndto 3rdlocation while Chl-a experienced a decrease of 20.57 μg/L from the 2ndto the 3rdlocation. Turbidity declined by 18.7 NTU from the 3rd to 4th location.

The averaged water quality parameters from eight locations offered a comprehensive representation of the pond's environmental conditions. These averages, coupled with minimal standard errors signifying precise measurements, provided valuable insights for environmental modeling. Serving as a baseline, the data facilitated the evaluation of water quality trends and potential environmental impacts. Specifically, the average temperature aligned with typical values for summer days, and the recorded pH falls within the anticipated range for pond environments. Both average TDS and turbidity values were within expected norms, while the levels of PC and Chl-a suggest the presence of blue-green algae in the pond.

The imaging sensing approach employed for this study involved visual observation to detect and monitor large-scale algae bloom distributions on water surfaces. This method aligns with established visual sensing practices commonly used in environmental monitoring. In parallel, water samples collected for analysis underwent measurement and characterization within a laboratory setting, employing advanced test equipment—a standard practice in water sample analysis. Furthermore, the real-time measurement of water parameters, such as temperature, pH, turbidity, and total dissolved solids (TDS), was conducted using sensors mounted on a UAV. The accuracy of these sensor measurements is crucial, and to assess their precision, calibrations were executed by comparing the sensor-derived results with those obtained from commercially available water parameter measurement tools. The calibration process results (linearity and R²) validated that the sensor measurements closely aligned with the outcomes of established commercial measurement tools, affirming the reliability and accuracy of the UAV-based real-time sensing system. The linear actuators performed flawlessly in the field test measurements, and no observable change occurred in their performance during the measurements.

### GIS Mapping, Heatmaps, and HAB Detection

True color and NIR band color Orthomosaic images from the STU system vividly depict the extensive distribution of algal and cyanobacteria blooms, particularly concentrated in the vicinity of the pond's coastline. This pattern highlights the ecological dynamics of the area, showcasing the likely interplay between water quality, nutrient levels, and environmental factors.

NIR imaging generated a clear visualization of algae and cyanobacteria distribution within the pond. However, due to the inherent challenge of distinguishing algal and cyanobacteria distributions from the background (water body), a Python algorithm was used to effectively highlight the algal bloom distributions and find the edges for a fully comprehensive view of the algal blooms.

The Python program loaded the image, converted it to the Hue Saturation Value (HSV) color space, and applied a color mask to extract algal and cyanobacteria based on their green color. It then performs Canny edge detection to identify edges within the extracted algal blooms and displayed both the original algal bloom images and their corresponding edge detection results. Cyanobacteria were detected using two thresholds: a lower green and an upper green. The lower green threshold corresponds to the minimum acceptable value for each component in the HSV color space. In this program, the HSV values used for lower green were 1, 100, and 100 respectively; the upper green could further refine the range of green colors considered HABs. The upper-green HSV values were 106, 255, and 255 respectively. The threshold values used for Canny were 20 and 100 for weak and strong edges respectively. The resulting edged and enhanced NIR image enhanced the readability of algal and cyanobacteria bloom distribution compared to NIR on its own. Parameter heatmaps were provided for the eight testing/sampling locations.

GIS mapping represents the parameter heatmaps at a depth of 1 m across the eight testing/sampling locations. These visualizations were generated utilizing an IDW Spatial Analyst tool in ArcGIS Pro software. Spatial differences could be observed for each parameter, suggesting that even within a relatively mixed waterbody small-scale variation is possible. These variations could easily be missed using traditional sampling techniques. Hence, real-time interpretation of data from the STU can be used to direct water collection locations. Viewing data on the fly can also allow future missions to fill in spatial data gaps between areas of distinct water quality. For example, the water temperature at testing/sampling location 2 was lower than at the other locations. Adding sensing data measurements between the western testing/sampling sites and testing/sampling site 2 can help resolve how water temperature shifts within the targeted area. When combined with enhanced NIR imagery of blooms, water collection, and sensing, efforts can be directed to better define the spatial extent of the bloom and the difference between water quality within and outside the bloom.

Reliable HAB monitoring is crucial to assess the impacts of algal overgrowth, with in-situ and STU measurements being a cost-effective choice. This study has developed and validated a LoRaWAN UAV-mounted STU capable of collecting samples and wireless real-time data transmission. This system provided a comprehensive platform for studying harmful algal blooms and water quality using water sample collection for sophisticated laboratory analysis, real-time water quality parameter sensing and transmission, multispectral imaging of the water body, and GIS mapping. Eight water samples were collected the target body of water (the pond) for PC and Chl-a variation analysis. Orthomosaic images were captured from the pond and merged into single true-color and NIR-band images. A GIS map was created and stored in a database. The was samplers indicate 100% activation success across all locations and performance remained intact.

The developed wireless sensing technology and overall STU system allow for water sampling and studying water conditions in remote locations in real time. Practical applications include, but are not limited to, monitoring remote bodies of water, reservoirs, lakes, swamps, bayous, and ponds, among others. Given semi-autonomous or fully autonomous flight capabilities, an STU system of the present disclosure can monitor the bodies of water at specific times during the year with minimum to no human input required.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A submersible testing unit (STU) system for testing a body of water, the STU system comprising:
an STU that includes:
a main body designed and configured to be at least partially submerged in the body of water;
a sampling system supported by the main body and that includes a plurality of sampling chambers designed and configured to receive a sample of the water in the body of water when the inlet is submerged in the water;
a sealing system, in operable communication with the opening, having a sealing member designed and configured to seal the inlet after receiving the sample of water, the sealing member having a sealed position and an unsealed position; and
a seal-actuation system operatively coupled to the sealing member and designed and configured to move the sealing member between the sealed and unsealed positions.

2. The STU system of claim 1, wherein the STU includes a plurality of sample chambers each having a corresponding inlet, wherein the sealing system is configured to unseal the inlets serially relative to one another.

3. The STU system of claim 2, wherein the sealing system includes a plurality of sealing members each associated with a corresponding one of the inlets.

4. The STU system of claim 3, wherein the seal-actuation system comprises a single actuator that actuates each of the plurality of sealing members.

5. The STU system of claim 4, wherein the single actuator is located centrally relative to the plurality of sealing members and each sealing member includes a strike member, and the seal-actuation system includes:
a moveable pusher that is designed and configured to push each strike member; and
a rotational motor having a rotational axis, wherein the rotational motor drives the moveable pusher about the rotational axis to serially push each of the strike members.

6. The STU system of claim 4, wherein the sealing system comprises:
a movable seal having:
an elongated seal region that, when the movable seal and the plurality of sample chambers are in a first position relative to one another, seals all of the inlets; and
a passthrough region adjacent to at least one portion of the elongated seal region; and
wherein the seal-actuation system designed and configured to move at least one of the moveable seals and the plurality of sample chambers relative to one another so as to selectively align the passthrough region with any one of the inlets so as to allow water to flow into the corresponding sample chamber when the inlet is submerged in the body of water.

7. The STU system of claim 3, wherein:
the sealing system comprises a plurality of movable seals corresponding, respectively, to the plurality of inlets; and
the seal-actuation system comprises a plurality of seal actuators corresponding, respectively, to the plurality of movable seals, wherein each of the plurality of seal actuators is designed and configured to open the corresponding inlet so as to allow water to flow into the corresponding sample chamber when the inlet is submerged in the body of water.

8. The STU system of claim 1, further comprising a base, wherein the main body is removably engaged with the base.

9. The STU system of claim 8, wherein the base includes at least one submersible sensor.

10. The STU system of claim 9, wherein the at least one submersible sensor is selected from the group consisting of a temperature sensor, a total-dissolved-solids sensor, a pH sensor, and a turbidity sensor, a depth sensor, a dissolved oxygen sensor, carbon dioxide sensor, a nutrient sensor, a fluorometer, and a heavy metal sensor.

11. The STU system of claim 1, further comprising:
a tether for connecting the STU from an aerial vehicle; and
a disconnect device coupled to the tether, wherein the disconnect device is configured to selectively disconnect the STU from the aerial vehicle.

12. The STU system of claim 11, wherein the disconnect device is configured to automatically disconnect the STU from the aerial vehicle when a tensile force on the tether exceeds a predetermined threshold value.

13. The STU system of claim 11, wherein the disconnect device is configured to respond to a remote triggering so as to disconnect the STU from the aerial vehicle.

14. The STU system of claim 11, wherein the disconnect device is configured to both automatically disconnect the STU from the aerial vehicle when a tensile force on the tether exceeds a predetermined threshold value and respond to a remote triggering so as to disconnect the STU from the aerial vehicle.

15. The STU system of claim 1, wherein the main body includes at least one watertight compartment containing:
control circuitry for controlling the STU; and
at least one battery for powering the seal-actuation system and the control circuitry.

16. The STU system of claim 15, further comprising a magnet-actuated power switch located within the at least one watertight compartment, wherein the magnet-actuated power switch is located and configured to be actuated by a magnet located externally relative to the at least one watertight compartment.

17. The STU system of claim 15, further comprising:
a charging dock having a first set of electrical contacts;
a battery charging system, located onboard the STU, for charging at least one of the at least one battery; and
a second set of electrical contacts located externally on the STU, wherein the second set of electrical contacts are electrically connected to the battery charging system and are configured to engage the first set of electrical contacts on the charging dock.

18. The STU system of claim 1, further comprising a tether that includes at least one cable, wherein the STU is configured to be coupled, via the tether, to an aerial vehicle containing:
control circuitry for controlling the STU via the at least one cable; and
at least one battery for powering the seal-actuation system via the at least one cable.

19. The STU system of claim 1, wherein each of the plurality of sampling chambers is removably secured to the main housing.

20. The STU system of claim 19, wherein each of the plurality of sampling chambers is removably secured to the main housing by a magnetic connection.
